(19) United States
(12) Patent Application Publication
(10) Pub. No.: US 2004/0010308 A1
(43) Pub. Date: Jan. 15, 2004

(54) Implantable composite device and corresponding method for deflecting embolic material in blood flowing at an arterial bifurcation

(75) Inventors: Nitzan Zafrir-Pachter, Karkur (IL); Ygael Grad, Tel Aviv (IL); Avraham Rapaport, Tel Aviv (IL); Boaz Nishri, Doar Na Menashe (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: MindGuard Ltd.

(21) Appl. No.: 10/615,945

(22) Filed: Jul. 10, 2003

Related U.S. Application Data
(63) Continuation-in-part of application No. PCT/IL02/00024, filed on Jan. 11, 2002.
(63) Continuation-in-part of application No. 09/637,287, filed on Aug. 11, 2000.
Application No. 09/637,287 is a continuation-in-part of application No. 09/484,965, filed on Jan. 18, 2000, now Pat. No. 6,348,063.

(30) Foreign Application Priority Data
Jan. 11, 2001 (IL) 140871

(51) Int. Cl. A61F 2/06

(52) U.S. Cl. 623/1.35; 623/1.22; 623/1.39

(57) ABSTRACT

An implantable device particularly useful for implantation in the common carotid artery at its bifurcation with the internal carotid artery and the external carotid artery for reducing the risk of a stroke, includes a base element for anchoring the device in the artery, and a deflector element for covering the inlet of the internal carotid artery. The deflector element is formed with openings of a size and configuration to deflect emboli in the blood to the external carotid artery without blocking blood flow through the external or internal carotid arteries. The deflector element is attached to a supporting portion of the base element to produce a composite construction. In one described embodiment, the base element is a coil having two opposing ends which overlap to permit expansion for deployment in the artery, and in a second described embodiment the base element is a tube expandable for deployment in the artery.

### RELATED APPLICATIONS

The present application is a continuation-in-part of PCT/IL02/00024, filed Jan. 11, 2002, which claims priority from Israel Patent Application No. 140,871 filed Jan. 11, 2001. This application is also a continuation-in-part of U.S. patent application Ser. No. 09/637,287, filed Aug. 11, 2000, which is a continuation-in-part of U.S. patent application Ser. No. 09/484,965 filed Jan. 18, 2000, now U.S. Pat. No. 6,348,063.

### FIELD AND BACKGROUND OF THE INVENTION

The present invention relates to implantable medical devices for deflecting embolic material in blood flowing through arteries, and, more particularly, to an implantable composite device and corresponding method for deflecting embolic material in blood flowing at an arterial bifurcation. The implantable composite device, herein, also referred to as the deflecting device, featuring a unique expandable dual diameter composite structure of an expandable base element, and, a deflector element, supported and anchored by the base element, for deflecting the embolic material while filtering the blood flowing at the arterial bifurcation, reduces the risk of embolic material entering the internal carotid artery of a subject, and, reduces the risk of blood clots occurring in the subject. Herein, embolic material and blood clots are collectively and interchangeably referred to as ‘embolic material’.

A major portion of blood supplied to the brain hemispheres is by two major arteries in the neck, referred to as common carotid arteries (CCA), each of which branches off, or bifurcates, into an internal carotid artery (ICA), and, into an external carotid artery (ECA). Blood to the brain stem is supplied by two vertebral arteries.

Stroke is a leading cause of disability, death, and health care expenditure. It is the second most common cause of death worldwide, exceeded only by heart disease, and is the third most common cause of death in the U.S., as described in Heart And Stroke Statistical Update, Dallas, Tex., USA, American Heart Association, 2000.

Stroke is caused either by ischemia-infarction or intracranial hemorrhage. Infarction constitutes 85 to 90 percent of the total group in western countries, as described by Sacco, R. L., Toni, D., and, Mohr, J. P., in Classification Of Ischemic Stroke, Stroke: Pathophysiology, Diagnosis And Management, editors: Barnett, H. J. M., Mohr, J. P., Stein, B. M., and, Yatsu, F. M., third edition, Churchill Livingstone, N.Y., USA, 1998, 271-83. The pathogenesis of ischemic stroke is complex with multiple potential mechanisms. The carotid plaque is only one source of stroke, accounting for no more than 15-20% of cases, as described by Petty, G. W., Brown, Jr., R. D., Whisnant, J. P., Sicks, J. D., O'Fallon, W. M., and, Wiebers, D. O., in Ischemic Stroke Subtypes, A Population-based Study Of Incidence And Risk Factors, Stroke, 1999, 30, 2513-16. More frequently, infarcts are caused by more proximal sources of emboli, that is, the heart and the aortic arch. The commonest causes of cardioembolic stroke are nonrheumatic (often called nonvalvular) atrial fibrillation, prosthetic valves, rheumatic heart disease (RHD), congestive heart failure, and ischemic cardiomyopathy.

A recent population based study from Rochester, Minn., USA, found that the main identifiable subtype of ischemic stroke was cardioembolic with nearly 30% of cases, while all large vessel cervical and intracranial atherosclerosis with stenosis altogether constituted about 16%, as described by Petty et al., ibid. Further, often multiple mechanisms coexist, as described by Caplan, L. R., in Multiple Potential Risks For Stroke, JAMA 2000, 283, 1479-80. Wilson, R. G. and Jamieson, D. G., in Coexistence Of Cardiac And Aortic Sources Of Embolization And High-grade Stenosis And Occlusion Of The Internal Carotid Artery, J. Stroke Cerebrovasc Dis., 2000, 9, 27-30, reviewed the experience of Petty et al. with patients who had high grade internal carotid artery stenosis or occlusion, and also had cardiac and aortic evaluation. Potential cardiac or aortic sources of emboli were present in 54% of patients; aortic arch plaques greater than 4 mm in diameter were found in 26% of patients with severe internal carotid artery occlusive disease.

Prevention is clearly the most cost-effective approach to decreasing the burden of stroke. Available strategies to prevent stroke include medical treatment, surgery (carotid endarterectomy), and carotid stenting.

Current medical treatments include antiplatelet drugs, such as aspirin, ticlopidine, clopidogrel, and dipyridamol, for presumed athreothrombotic origin. These treatments reduce the risk for recurrent ischemic event by no more than 15-20%. Anticoagulants, such as Warfarin for non valvular atrial fibrillation, reduce the risk by 60%, however, even in carefully conducted and monitored clinical trials, a substantial number of patients stopped anticoagulation, as described by Hart, R. G., Benavente, O., McBride, R., and, Pearce, L. A., in Antithrombotic Therapy To Prevent Stroke In Patients With Atrial Fibrillation: A Meta-analysis, Ann Intern Med., 1999, 131, 492-501.

Carotid endarterectomy was shown to be beneficial in selected cases of medium grade symptomatic, and also in asymptomatic carotid stenosis, by greater than 60%, whenever complication rates are kept low, as described by Chassin, M. R., in Appropriate Use Of Carotid Endarterectomy (editorial), N. Engl. J. Med., 12998, 339, 1468-71. Nevertheless, a high proportion of recurrent stroke was not related to the large artery atherothrombotic disease, but to other causes including cardioembolism, as recently reported by the NASCET (North American Symptomatic Endarterectomy Trial) investigators, Barnett, H. J. M., Gunton, R. W., Eliasziw, M., et al., in Causes And Severity Of Ischemic Stroke In Patients With Internal Carotid Artery Stenosis, JAMA, 2000, 283, 1429-36. In fact, strokes related to cardioembolism tended to be more severe. The population of patients with carotid stenosis in ‘real life’ often includes patients with severe cardiac disease, concomitant protruding aortic arch atheroma, atrial fibrillation, or congestive heart failure. The proportion of patients with such concomitant disease increases substantially in an elderly population. Thus, the risk of recurrent cardioembolic stroke, even in patients operated for carotid stenosis, is estimated to be substantially higher, as described by Barnett, H. J. M., et al., ibid.

Carotid artery stenting has potential advantages of offering treatment to high risk patients with carotid stenosis, lowering peri-procedural risk, decreasing costs, and reducing patient inconvenience and discomfort. Preliminary results from clinical trials comparing carotid stenting to carotid endarterectomy have shown similar results, as described in Major Ongoing Stroke Trials, Stroke, 2000, 31, 557-2.

The approach to prevention of such a multi factorial complex syndrome as stroke is necessarily multifaceted. Carotid angioplasty, with stenting by itself, does not address additional sources of emboli, even after successful reduction of local stenosis. More efficient endovascular approaches to stroke prevention needs to take into account this complexity in cerebrovascular disease. In this context, an intravascular implant that also addresses prevention of emboli from proximal sources can be a valuable addition in the arsenal of the practicing physician.

Introducing filtering means into blood vessels, particularly into veins, has been known for some time. However, filtering devices known in the art are designed for filtering blood flowing in the vena cava, and for stopping embolic material having a diameter of the order of centimeters, but, are unsuitable to deal with arterial embolic material, with which the present invention is concerned, especially in cases where the diameter of such material is typically of the order of down to microns. Furthermore, the flow of blood in the veins does not resemble arterial flow by its hemodynamic properties. However, when considering the possible cerebral effects of even fine embolic material occluding an artery supplying blood to the brain, the consequences may cause irreversible brain damage, or, may even be fatal.

In light of the short period of time during which brain tissue can survive without blood supply, there is significant importance to providing suitable means for preventing even small sized embolic material from entering the internal carotid artery, so as to prevent brain damage, or, even death.

The size of the filaments that make up the deflector element, and the Porosity Index thereof, defined hereinafter, are major features of the deflecting device of the present invention, as explained herein, below. By contrast, in venous blood filters currently known in the art, no particular attention has been given to the size of the filaments. It is noted that embolic material in venous blood is made up of only blood clots, while in arterial blood, it is necessary to deal with emboli featuring different materials, such as blood clots and atherosclerotic plaque debris, etc.. Accordingly, in order to provide efficient filtering means, a blood deflector element should be of fine mesh. However, a fine mesh blood filter has a higher tendency toward occlusion.

It is also be noted that the flow ratio between the ICA and the ECA is about 3:1-4:1. This flow ratio indicates the significantly higher probability of embolic material flowing into the ICA rather than into the ECA. However, the ECA is a relatively non-hazardous artery because it supplies blood to superficial organs in the face and head, which are not life supporting and which receive blood supply from collateral blood vessels. Therefore, embolic material reaching these organs does not cause substantial damage to a subject.

Manufacturing braided stents and prostheses is known in the art. For example, in the disclosures of U.S. Pat. No. 6,083,257, U.S. Pat. No. 5,718,159, U.S. Pat. No. 5,899,935, and, U.S. Pat. No. 6,494,907, the teachings of which are incorporated by reference as if fully set forth herein, there are described methods of manufacturing braided stents. Such braided stents present various advantages. However, they are all made for the purpose of preventing stenosis and for supporting blood vessels. The relatively large mesh sizes employed, and, the thickness and shape of the stent struts, make them unsuitable for use as a deflector element for deflecting embolic material.

The above-cited related patent application Ser. Nos. 09/637,287 and 09/484,965 (the latter having issued as U.S. Pat. No. 6,348,063), as well as PCT Application PCT/IL00/00145 (the latter being equivalent to patent application Ser. No. 09/950,027) discloses implantable devices implantable in an artery at a bifurcation into a first branch supplying blood to a vital region having a high sensitivity to emboli in the blood, and a second branch supplying blood to a less vital region having a lower sensitivity to emboli in the blood, for deflecting emboli in the blood to the second branch without blocking blood flow through the second branch or through the first branch. Such implantable devices were described particularly for implantation in the CCA to deflect emboli in the blood to the ECA without blocking blood flow through the ECA or through the ICA.

### OBJECT AND BRIEF SUMMARY OF THE INVENTION

An object of the present invention is to provide, in an implantable device of the type described in the above-cited patent applications, improvements which simplify the construction, reduce the cost of manufacture, and/or increase the flexibility of the device for internal positioning.

According to one aspect of the present invention, there is provided an implantable device implantable in an artery of a patient at a bifurcation thereof into a first branch supplying blood to a vital region having a high sensitivity to emboli in the blood, and a second branch supplying blood to a less vital region having a lower sensitivity to emboli in the blood; the implantable device being of tubular configuration initially of a small diameter for facilitating its introduction into and deployment through the artery to the bifurcation, and expandable to a larger diameter for implantation in the artery and the bifurcation; the implantable device comprising: a base element configured and dimensioned for anchoring the implantable device in the artery at the bifurcation; and a deflector element configured and dimensioned for covering the inlet of the first branch at the bifurcation when the implantable device is implanted in the artery; the deflector element being formed with openings therethrough of a size and configuration to deflect emboli in the blood to the second branch without blocking blood flow through the second branch or through the first branch; the base element including an anchoring portion engageable with the inner surface of the artery for anchoring the implantable device therein, and a supporting portion engageable with a surface of the deflector element for fixing the deflector element over the inlet of the first branch at the bifurcation when the implantable device is anchored in the artery; the deflector element being attached to the supporting portion of the base element to produce a composite construction.

In one described preferred embodiment, the base element is a coil having two opposing ends which overlap in its initial small diameter condition. More particularly, in the described preferred embodiment, the coil is a perforated sheet coiled into the tubular configuration.

In a second described preferred embodiment, the base element is a tube in its initial small-diameter condition expandable to the larger-diameter condition.

According to further features in some described preferred embodiments, the supporting portion of the base element includes a plurality of spaced parallel filaments extending longitudinally of the base element. In addition, the supporting portion of the base element further includes a pair of additional filaments extending circumferentially of the base element on opposite ends of the supporting portion.

In one described preferred embodiment, the deflector element is supported on, and is draped between, the plurality of spaced parallel filaments of the supporting portion of the base element in the small-diameter condition of the implantable device. In a second described preferred embodiment, the deflector element is supported on, and is stitched to, the plurality of spaced parallel filaments of the supporting portion of the base element in the small-diameter condition of the implantable device and in a third described preferred embodiment, the deflector element is supported on, and is wrapped around, the plurality of spaced parallel filaments of the supporting portion of the base element in the small-diameter condition of the implantable device.

According to a further feature in one described preferred embodiment, the deflector element includes a finely-meshed area circumscribed by an unmeshed frame. Preferably, in all the described preferred embodiments, the base element and the deflector element are both of a meshed structure in which the meshed structure of the base element has a larger porosity index than that of the deflector element.

As indicated earlier, the invention is particularly useful for implantation in the common carotid artery for reducing the risk of a stroke. Accordingly, in the described preferred embodiments, the implantable device is configured and dimensioned for implantation in the patient's common carotid artery at its bifurcation with the internal carotid artery constituting the first branch, and the external carotid artery constituting the second branch.

According to still further features in the described preferred embodiments, the openings in the deflector element are within the range of 100 μm to 700 μm, preferably within the range of 100 μm to 400 μm. In addition, the base element is constituted of wire of a diameter within the range of 100 μm to 1500 μm, and the deflector element is constituted of wire of a diameter within the range of 20 μm to 75 μm. Preferably, the base element is constituted of wire of a diameter within the range of 100 μm to 200 μm, and the deflector element is constituted of wire of a diameter within the range of 20 μm to 75 μm.

According to another aspect of the present invention, there is provided a method of reducing the risk of a stroke in a patient, comprising: providing an implantable device as described above, configured and dimensioned for implantation in the patient's common carotid artery at its bifurcation with the internal carotid artery constituting the first branch, and the external carotid artery constituting the second branch; and implanting the implantable device in the patient's common carotid artery at the bifurcation.

According to a still further aspect of the present invention, there is provided a method for assembling an implantable device implantable in an artery of an individual at a bifurcation thereof into a first branch supplying blood to a vital region having a high sensitivity to emboli in the blood, and a second branch supplying blood to a less vital region having a lower sensitivity to emboli in the blood: selecting a base element of tubular configuration, the base element having states of different diameter, a first state being a small diameter for facilitating its introduction into and deployment through the artery to the bifurcation, and expandable to a second state being a larger diameter for implantation in the artery and the bifurcation; selecting a separate tubular deflector element comprised of elastic elements, the tubular deflector element being configured and dimensioned to cover the inlet of the first branch at the bifurcation when the implantable device is implanted in the artery, the unstretched diameter of the tubular deflector element being smaller than the diameter of the second state of the base element; inserting the base element in a contracted condition into the tubular deflector element; contracting the base element and the tubular deflector element to the first state thus facilitating its introduction into and deployment through the patient body to the bifurcation; and expanding the base element to the second state thus implantating the implantable device in the artery at the bifurcation and securing the separate tubular deflector element to the base element.

Further features and advantages of the invention will be apparent from the description below.

### BRIEF DESCRIPTION OF THE DRAWINGS

The invention is herein described, by way of example only, with reference to the accompanying drawings wherein:

FIG. 1A is a perspective view of a base element for a deflecting device constructed in accordance with the present invention;

FIG. 1B is a deflecting device, similar to that of FIG. 1A, constructed in accordance with the present invention;

FIG. 2A is a deflecting device similar to that of FIG. 1A, in which the supporting portion for the deflector element has been removed;

FIG. 2B is a deflecting device similar to that of FIG. 1B, in which the supporting portion for the deflector element has been removed;

FIG. 3A is a perspective view of a base element for a deflecting device in accordance with another preferred embodiment of the present invention;

FIG. 3B is an illustration of a grid structure used to produce the deflecting device of FIG. 3A;

FIGS. 4A, 4B, and 4C are cross-sections of the deflecting device of FIG. 3A along the A-A plane and illustrate the insertion and positioning of the device according to a preferred embodiment of the present invention;

FIG. 4A schematically shows the deflecting device of FIG. 3A in collapsed form prior to expansion;

FIG. 4B shows the deflecting device of FIG. 3A in an expanded form in a first operative position;

FIG. 4C shows the deflecting device of FIG. 3A in an expanded form in a second operative position;

FIGS. 5A and 5B are schematic illustration of two deflector elements, according to two alternative preferred embodiments of the present invention;

FIG. 5A shows a sheet-like deflector element;

FIG. 5B a tubular deflector element;

FIG. 6A is a schematic illustration of the deflector element of FIG. 5A, coupled to a base element of FIG. 1A or 2A or 3A, in accordance with the present invention;

FIG. 6B is a schematic illustration of the deflector element of FIG. 5A, coupled to a base element of FIG. 1B or 2B;

FIGS. 7A, 7B, and 7C illustrate a method of folding the deflecting device when inserting it into the artery, in accordance with the present invention;

FIG. 7A shows a cross section of the deflecting device in its collapsed form;

FIG. 7B shows a cross section of the deflecting device in a partially expanded state;

FIG. 7C shows a cross section of the deflecting device in its fully expanded form;

FIGS. 8A, 8B, and 8C illustrate the introduction of the deflecting device and its deployment within the artery;

FIG. 8A shows the deflecting device in its collapsed state;

FIG. 8B shows the deflecting device in a partially expanded state;

FIG. 8C shows the deflecting device in its expanded state;

FIG. 9A schematically illustrates the deflecting device of FIG. 6A, located in the bifurcation zone of the carotid artery;

FIG. 9B schematically illustrates the deflecting device of FIG. 6B located in the bifurcation zone of the carotid artery;

FIG. 10 is a cross section showing a deflecting device composed of a base element of FIG. 3A and a deflector element of FIG. 5B located in the bifurcation zone of the carotid artery;

FIGS. 11A and 11B show how the deflecting devices of the invention can be used in the treatment of aneurysms;

FIG. 11A shows a deflecting device of FIG. 6A at the site of an aneurysm; and

FIG. 11B shows the deflecting device of FIG. 6B at the site of an aneurysm of another type.

It is to be understood that the foregoing drawings, and the description below, are provided primarily for purposes of facilitating understanding the conceptual aspects of the invention and various possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF PREFERRED EMBODIMENTS

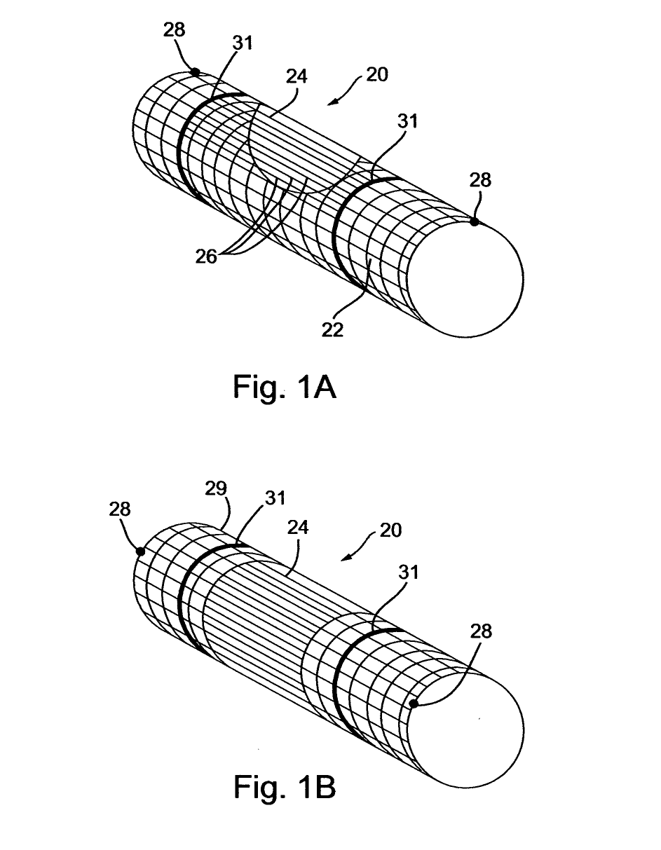

The implantable composite devices, or deflecting devices, described below are composed of two separate (principle) elements: (a) a base element, and, (b) a deflector element. Each of these elements can be produced in several forms (illustrative and non-limitative examples of which follow) and than interchangeably assembled to make the composite deflecting device. A base element for a deflecting device in accordance with a preferred embodiment of the present invention, generally designated 20, is shown in FIG. 1A. The base element of the deflecting device is made of fine wire manufactured into a net-like device having a construction suitable for expanding from a contracted position in which it is deployed through the vasculator of an individual, and expanded by means well known in the art, for example, by a balloon device coupled with a catheter. Alternatively, the base element of the device can be self-expandable, as is customary in the art with respect to peripheral stents. These techniques are well known to the skilled person, and are therefore not discussed herein in detail, for the sake of brevity.

The base element of the deflecting device 20 has an essentially cylindrical shape with its body 22 generally serving as an anchoring portion. An anchoring portion is a portion of the device that firmly contacts the walls of the artery. Such contact causes a proliferation of cells of the wall of the artery into the net of the device, and strongly anchors it to the artery thus preventing its accidental displacement. The physiological processes leading to such anchoring are well known in the art, and will therefore not be discussed herein in detail, for the sake of brevity. The net that makes up the anchoring portion 22 of the base element of the device can be of a large mesh, since it has no obstructing or filtering purposes.

A supporting portion 24 is provided, in this particular preferred embodiment of the present invention, to support the deflector element, as will be discussed hereinafter. It is constructed by a plurality of wires 26, parallely extending along the longitudinal axis of the device. The supporting portion 24 is integral with or attached to the anchoring portion 22.

The size and shape of the base element composited with the deflector element is adjusted to match the inlet of the internal carotid artery as will be further explained hereinafter.

The embodiment of FIG. 1B is similar to that of FIG. 1A. However, the base element of the deflecting device 20, which is essentially cylindrical, comprises a supporting portion 24 which is not limited to a part of the circumference of the device, as is the supporting portion 24 of FIG. 1A, but rather covers the whole circumference of the device. This arrangement, of course, is easier to use, inasmuch as, as will become apparent hereinafter, there is no need to exactly match the limited area of the deflecting portion with the opening of the ICA. Furthermore, two markers 31 (which in the particular embodiment of FIG. 1B are circular in shape) are provided, which are radio opaque and serve to aid a physician in the proper positioning of the device within the artery. The markers are visible under radiographic equipment. Other markers can also be provided, as will be apparent to the skilled person, such as gold points which may be used to position the device also with respect of its rotation around its axis or the beads 28 shown in FIGS. 1A and 1B, 2A and 2B, 3A and 3B, 6A and 6B, and 8A, 8B, and, 8C. In the case of devices of the type shown in FIGS. 1A, 2A, and 3A, the markers must be attached to the device in such a position that the center of the deflecting area can be accurately located.

The structure of the anchoring portion 29 (FIG. 1B) and of the supporting portion 24 is essentially similar to the structure illustrated with reference to FIG. 1A.

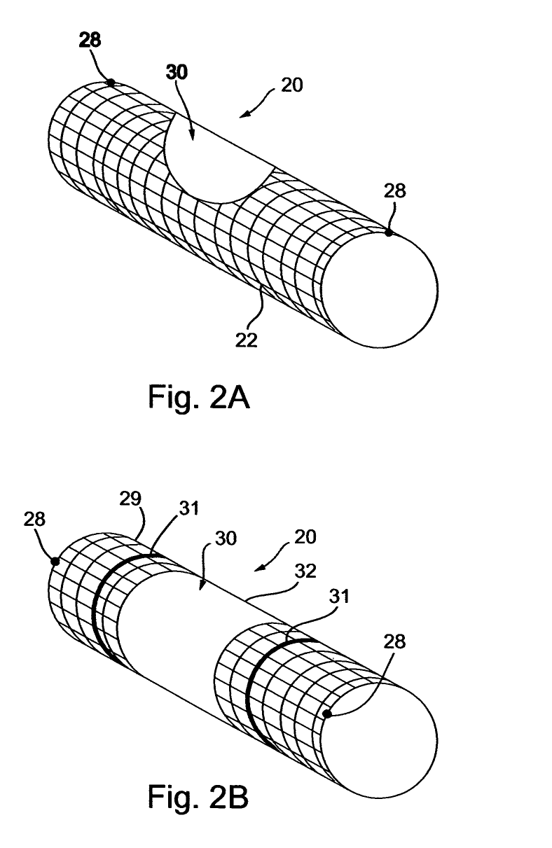

Looking now at FIG. 2A, a device similar to that schematically shown in FIG. 1A is shown. This device differs from that of FIG. 1A in that the supporting portion 24 thereof has been omitted, leaving a gap indicated at 30. Similarly, the device of FIG. 2B differs from that of FIG. 1B in that the supporting portion 24 of FIG. 1B has been omitted, leaving a gap indicated at 30. Two or more supporting rods 32 can be provided in this particular embodiment of the invention, to keep both ends of device 20 connected. The omission of the supporting portion is possible inasmuch as the deflector element will be superimposed to the body of the basis of the deflecting device, and will thus be supported by it.

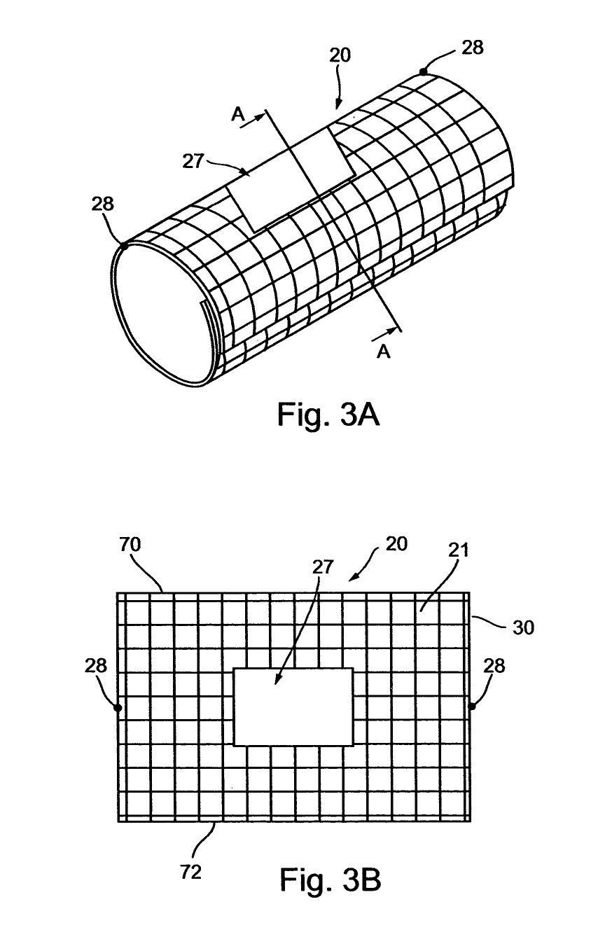

A further preferred embodiment of the device of the invention is shown in FIG. 3A. This particular embodiment utilizes a coiled base element to which the deflector element is attached. The device of FIG. 3A is constructed from the grid structure shown in FIG. 3B. This grid has an outer essentially rigid frame 30 with a meshed structure 21 attached. The size of this meshed structure and its mesh dimension are not important. They may be of any suitable type, shape, and size (for example, as used in conventional coronary stents) as long as they allow the device to function in its dual role as anchoring portion and support portion for the filtering element.

One area of the meshed structure 21 is an open zone 27, which will be covered with a deflecting filtering element as described below.

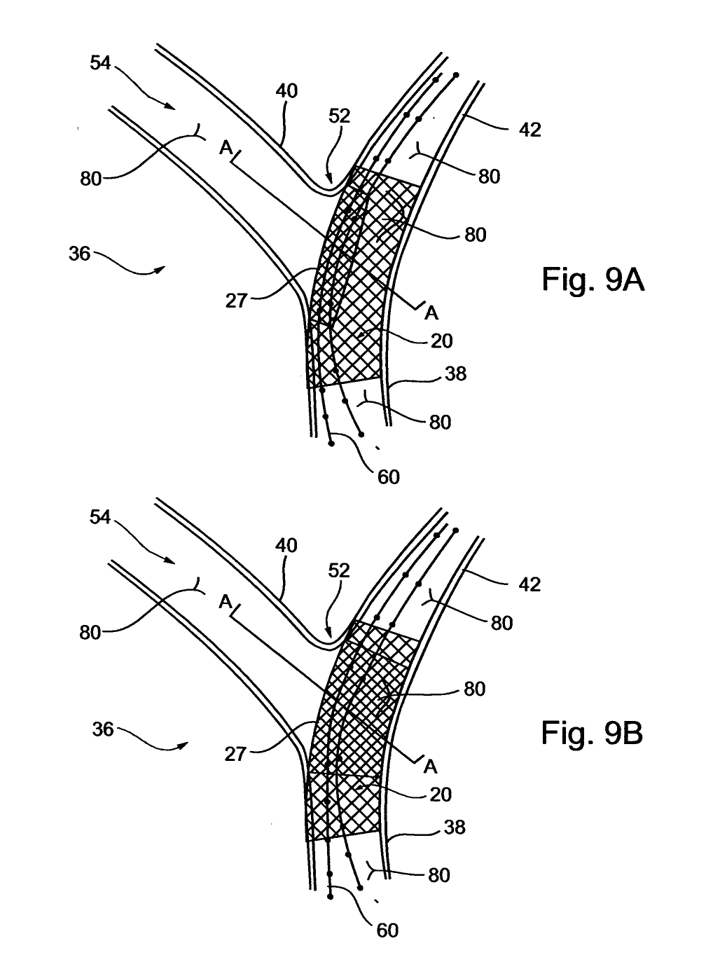

In another preferred embodiment of the invention, the filaments of 21 that lie within zone 27 are removed resulting in an open zone (this is the embodiment that is shown in FIGS. 3A and 3B). This zone 27 is the region which, when the device is coiled and introduced into the artery, is positioned in front of inlet 54 of junction 52 leading to artery 40 (FIGS. 9A and 9B).

The actual shape and size of zone 27 is not important, as long as care is taken to dimension it such that it will cover the entire junction.

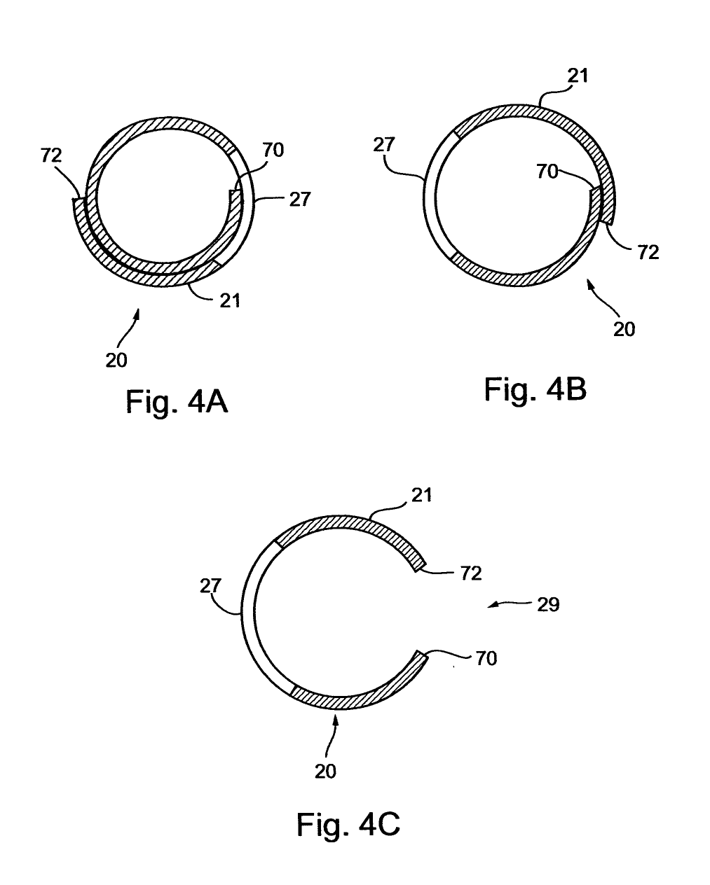

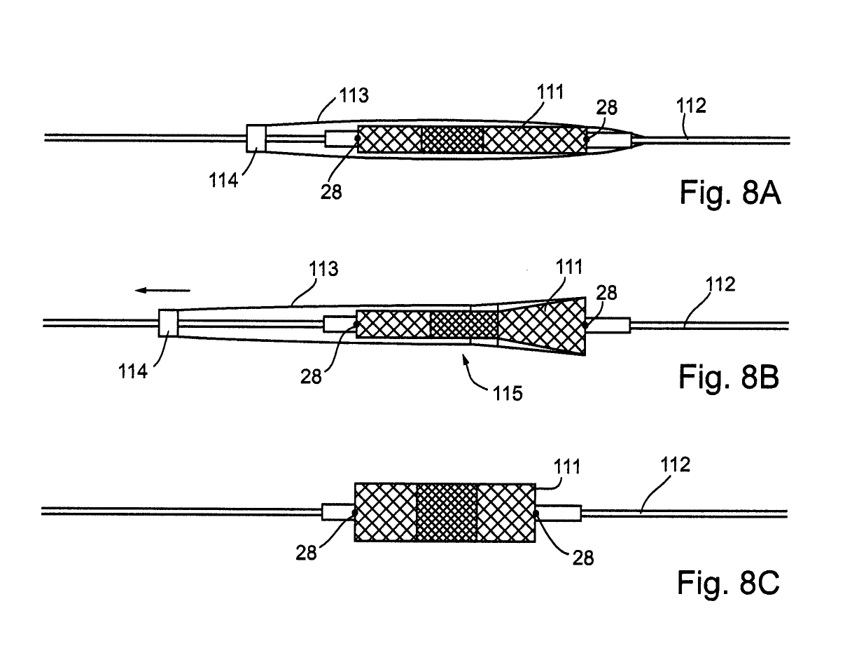

The device 20 is made of a material having an elasticity suitable for expanding from a contracted position in which it is deployed through the vasculator of an individual, and expanded by means well known in the art, as will be further explained hereinafter with reference to FIGS. 8A-8C. The device is schematically shown, in the coiled position in which it is deployed, in FIGS. 4A-4C, which depict a cross-section of FIG. 3A taken along the A-A plane. In the situation depicted in FIG. 4A, the device 20 is fully coiled, so that its total diameter is substantially smaller than that of the device in is expanded position. In this position end portions 70 and 72 do not necessarily need to be close to one another, and may be far apart, as shown in the figure.

FIG. 4B is a cross-section showing how the device of FIG. 3A would look in an expanded form in a first operative position; in the case in which the diameter of the artery is smaller than that of the fully expanded device.

FIG. 4C illustrates yet another situation, in which the diameter of the blood vessel where the deflecting device is to be positioned is greater than that of the fully expanded device. In this situation end portions 70 and 72 of device 20 do not overlap at all, and a gap 29 is formed between them. This situation is permissible, as long as the gap lies against a wall of the blood vessel. This further illustrates the flexibility of the device of the invention, which can be used in conjunction with various blood vessel diameters, and can adjust itself to unpredictable situations during deployment.

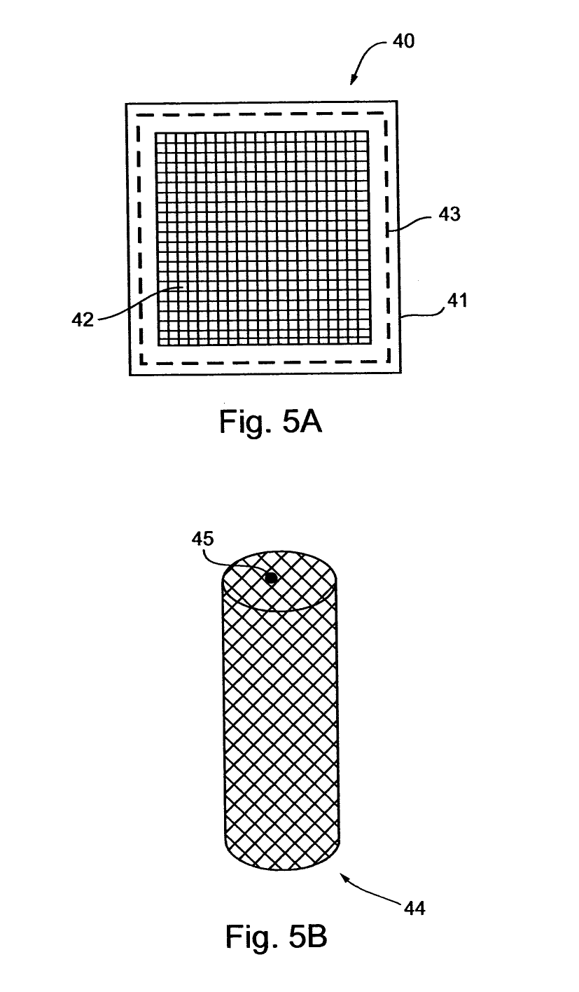

FIGS. 5A and 5B schematically illustrate two deflector elements according to two alternative preferred embodiments of the invention. In the embodiment of FIG. 5A a flat, sheet-like element 40, is provided, which is made of a frame 41 and of a finely meshed area 42. This is the area that will cover gaps 30 or 27 (FIGS. 2A, 2B, 3A, and 3B), or support portions 24 or 21 (FIGS. 1A and 1B and 3A (not shown in zone 27)). Element 40 may be attached to the body 22 or 29 or 21 (FIG. 1A, 2A or 1B, 2B or 3A, respectively) in any suitable way, for example, by stitching it with stitches 43 to the body.

In the embodiment of FIG. 5B, deflector element 44 is tubular in shape. Thus, the base element of the deflecting device is inserted into the longitudinal passageway 45 of tubular element 44. Both the base element and the deflecting device are collapsed on the delivery device (as is shown in FIG. 8) and then allowed to expand together, after they have been placed in the proper position in the artery. Proper contact between the deflecting device and the base element (on the one hand) and between the base element and the wall of the artery (on the other hand) is assured by a two step process. Firstly, the diameter of the basis is chosen, taking into account the diameter of the artery at the place at which the device will be installed. Secondly, the wires of which element 44 is manufactured are slightly elastic, and thus, the diameter of tubular element 44 is chosen to be slightly smaller than the maximum expanded diameter of the basis in the artery, thus stretching the deflector element over the base element. The insertion of the base element into the deflector element of FIG. 5B in order to obtain the composite device of the invention is not illustrated in the figures, for the sake of brevity.

It should be obvious to the person of experience, that many different well-known methods can be employed to produce either woven or non-woven material for the deflecting/filtering elements.

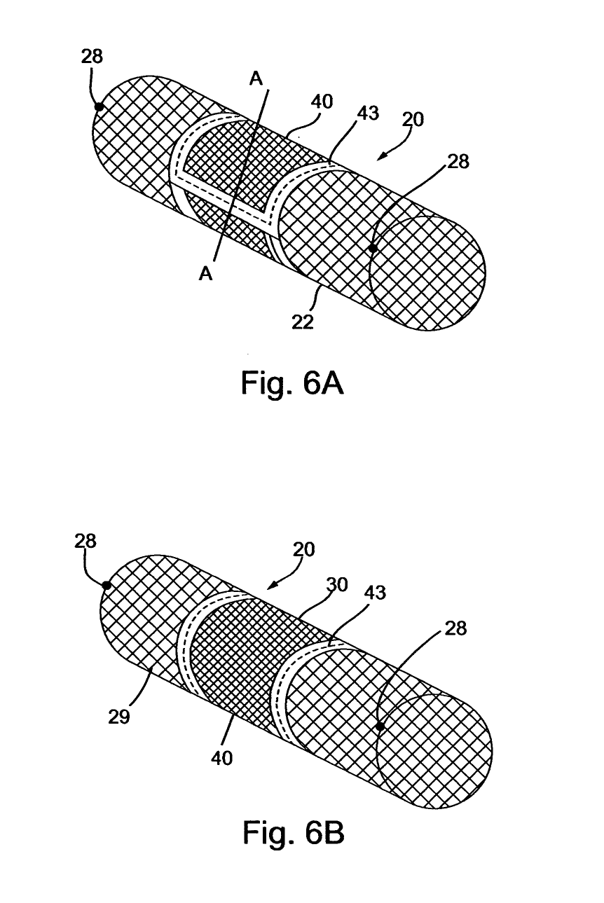

FIGS. 6A and 6B schematically illustrate the assembly of the deflecting device. In FIG. 6A the deflector element 40 of FIG. 5A has been stitched on to the device of FIG. 1A or 2A or 3A so as to cover the supporting portion 24 (FIG. 1A), or the gap 30 (FIG. 2A), or the supporting portion or gap in zone 27 of FIG. 3A. Likewise, in FIG. 6B the deflector element 40 of FIG. 5A has been at least partially stitched onto the basis in order to cover the supporting portion 24 (FIG. 1B), or the gap 30 (FIG. 2B). It can be seen by the skilled observer that, in the case shown in FIG. 6B, it is not necessary to stitch deflecting and filtering 40 to the base element; but, merely to wrap it around. The deflector element will be held in place by the force of the base element pressing against the walls of the artery.

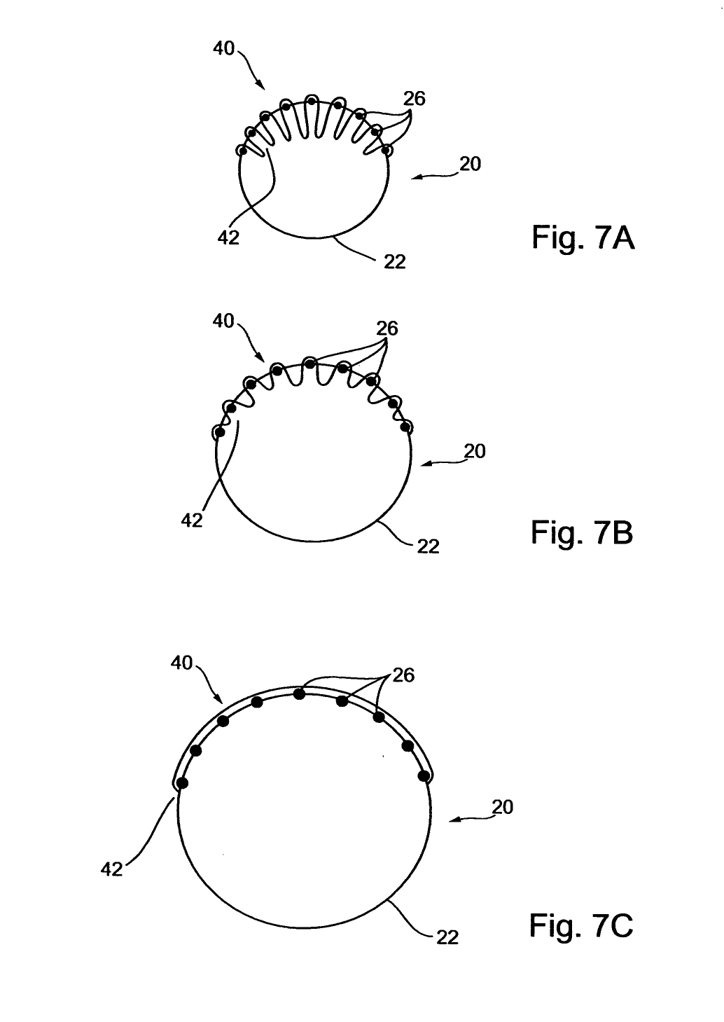

FIGS. 7A-7C illustrate a method of folding the deflecting device prior to deployment. As a non-limiting example, the case in which the deflector element of FIG. 5A is attached to the basis of FIG. 1A to form the device of FIG. 6A will be considered. In this example, the gap in the basis is one half of its circumference. FIG. 7A shows the device in its collapsed form. Here the finely meshed area 42 of the deflector element 40 is draped over and between the wires 26 that form the support area of the basis 20. As the device expands, the folds of the deflector element are drawn outwards as shown in FIG. 7B until the deflector element is stretched tightly over the surface of the fully expanded basis as shown in FIG. 7C.

Introduction of the device of the invention and its deployment are illustrated in FIG. 8. As will be apparent to the skilled person, using a self-expandable device is more convenient in many cases, because of the great mobility of the neck of the patient. The self-expandable device, of course, provides for a better anchoring of the device and is less likely than a balloon-expanded device to be dislodged in case of trauma.

FIG. 8A shows the device in its folded state, FIG. 8B shows it during the first stage of expansion, and, FIG. 8C shows it in fully expanded state. For purposes of illustration, the device 111 is the same device that is shown in FIG. 7. It is composed of base element 20 of FIG. 1A and the deflector element 40 of FIG. 5A. FIGS. 7A, B, and, C, correspond to the situation shown in FIGS. 8A, B, and, C, respectively. Device 111 is supported on a guide wire 112, which is used to introduce and guide it to the desired location. In its folded position, device 111 is covered with a covering envelope 113, which may be made of polymeric material, which keeps it in its folded state. Envelope 113 is connected to a retraction ring 114, which can be pulled away from device 111 by means not shown in the figure and well known to the skilled person. Looking now at FIG. 8B, when ring 114 is pulled away in the direction of the arrow, envelope 113 is pulled away with it, uncovering a portion of the device, indicated at 115. Since the envelope no longer obliges this portion 115 to remain in the folded position, and since the normal position of the device is expanded, this portion starts expanding to its natural, expanded state. This process is completed in FIG. 8C, when the envelope has been completely removed and the device is in its fully expanded position.

In the normally operative, expanded state, for example, as illustrated in FIG. 4C, radially directional elastic forces of the expandable property of expandable base element operate to keep the base element and therefore, the deflecting device, expanded, whereby, anchoring of the deflecting device in its location is less susceptible to undesired displacement as compared to deployment of balloon expanded stents. Following completion and positioning of the deflecting device 111, guide wire 112 is withdrawn from the subject, as in any other similar stent deployment procedure.

As will be apparent to the skilled person, the device of the invention does not necessarily need to be self-expandable, and it can be made of material that is expandable under pressure. In this case, the deployment of the device is carried out as in conventional stents, by placing the coiled device around an expandable balloon and by expanding the balloon under pressure when reaching the desired location. An expandable balloon can also be used in conjunction with a self-expandable device. In this case the balloon is used to increase the contact of the device with the inner walls of the arteries. These are conventional procedures and are, therefore, not illustrated in the figures, for the sake of brevity.

FIGS. 9A and 9B include illustration of a carotid artery portion, generally designated 36, in which the common carotid artery (CCA) is designated 38, the internal carotid artery (ICA) is designated 40, and, the external carotid artery (ECA) is designated 42. Blood, generally referenced by 80, flowing throughout carotid artery portion 36 is indicated in FIGS. 9A and 9B by the space between all other designated arteries and deflecting device elements and components.

FIGS. 9A and 9B show the devices of FIGS. 6A and 6B, respectively, in position in the bifurcation zone of the carotid artery. FIG. 9B also illustrates a device that could be comprised of any of the basis of FIG. 1, 2, or 3 inserted into the deflector element of FIG. 5B. By using suitable imaging equipment, the assembly consisting of the deflecting device mounted on a catheter was inserted through the vasculator of an individual, into the CCA, until the deflecting device 20 was positioned within the bifurcation zone 52, with the deflector element 27 extending opposite inlet 54 of ICA 40. In this position, the device was expanded whereby the anchoring walls of the deflecting device 20 anchor against respective inner walls of the common carotid artery 38 and the external carotid artery 42, with the deflector element 24 extending across inlet 54 of the internal carotid artery 40. Then, the catheter was removed via the vasculator of the individual, and the deployment of the deflector element 20 completed, as illustrated in FIGS. 9A and 9B. In this position, embolic material, which is schematically illustrated as particles flowing along flow lines 60 in FIGS. 9A and 9B, flow in the common carotid artery 38, and upon meeting the deflector element 24 they are prevented from entering the ICA 40, because their size is larger than the mesh of deflecting portion 24, and they are thus deflected into the external carotid artery 42.

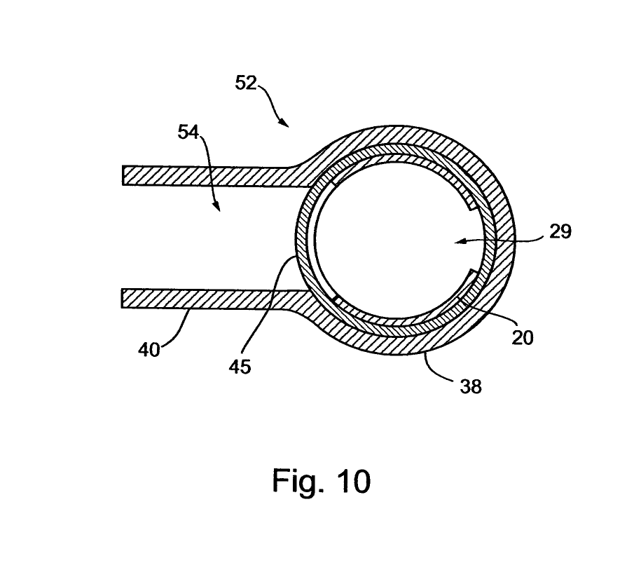

FIG. 10 is a cross-section taken along the AA plane of FIG. 9B. It illustrates a composite device made up by inserting the basis (20) of FIG. 3A into the tubular shaped deflector element (45) of FIG. 5B. Despite the gap 29 that results because the diameter of the artery is greater than the diameter of the fully expanded basis, the flexibility of the device enables it to adjust to the situation and to perform its intended function as is clearly shown in FIG. 10. The devices of the invention are well suited to the treatment of aneurysms.

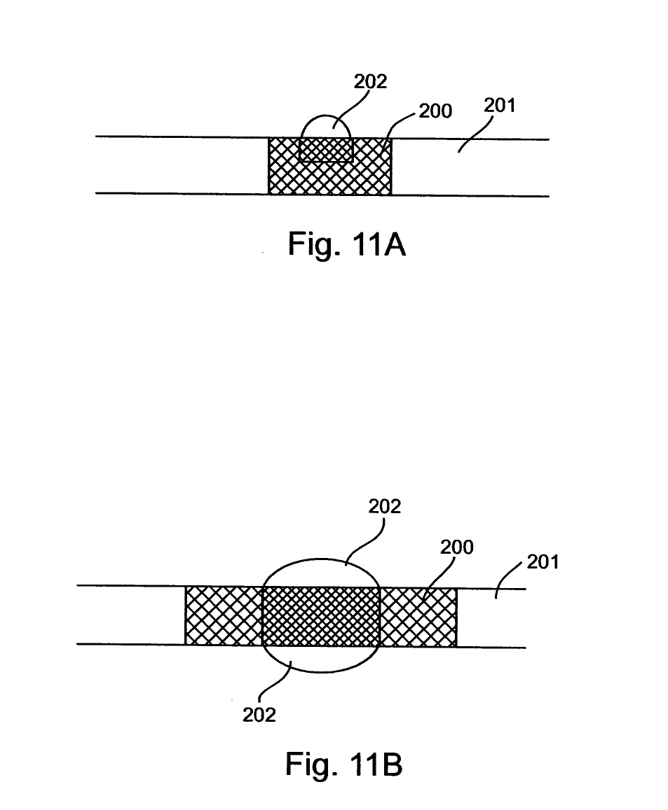

FIG. 11A shows a typical illustrative example of how a device 200 of the invention could be placed in a body lumen 201 in order to treat an aneurysm 202. Another type of aneurysm is shown in FIG. 11B in which the various elements are indicated by the same numerals as in FIG. 11A. Of course any combination of basis and deflector elements from among the many embodiments of the device of this invention could be chosen according to the specific requirements of the case. As will be seen by the skilled person, the porosity of the deflector element must be chosen in order to reduce the pressure on the aneurysm and will depend on the medical procedure that is carried out at the time of implanting the device. These procedures are well known to the skilled person and will not be herein described, for the sake of brevity.

The deflecting device of the present invention can be constructed in a way very similar to conventional stents. A person having ordinary skill in the art is knowledgeable of the various materials and methods suitable to make the deflecting device of the present invention. For instance, the deflecting device can be made of a material selected from the group consisting of nitinol, polymeric material, stainless steel, and, combinations thereof.

Preferably, the deflector element of the deflecting device has openings, preferably, in a range of between about 100 μm to about 700 μm, and, more preferably, in a range of between about 100 μm to about 400 μm, in order to effectively prevent an undesirable amount of dangerous embolic material flowing in the blood, from entering the internal carotid artery in the region of an arterial bifurcation. The diameters of the expandable dual diameter deflecting device may somewhat vary, according to actual conditions associated with embolic material, of different subjects. Preferably, the first diameter of the deflecting device in the closed position or contracted state varies, preferably, in a range of between about 1 mm to about 4 mm, and, more preferably, in a range of between about 1 mm to about 3 mm, and, the second diameter of the deflecting device in the open position or expanded state varies, preferably, in a range of between about 5 mm to about 35 mm, and, more preferably, in a range of between about 5 mm to about 30 mm.

Thickness and diameter of wire making up the base element of the deflecting device is preferably, in a range of between about 100 μm to about 1500 μm, and, more preferably, in a range of between about 100 μm to about 200 μm, while that of wire used for constructing the deflector element is preferably, in a range of between about 20 μm to about 75 μm, and, more preferably, in a range of between about 20 μm to about 40 μm. Of course, the entire deflecting device can also be constructed using wire of the same dimensions as that of the deflector element, whereby there would be no difference in mesh size between the body, that is, the base element of the deflecting device and the deflector element, in which case, a strengthening mechanism, for example, ribs, may be required for proper performance during normal operation for treating a subject.

The deflector element of the deflecting device of the present invention preferably fulfills certain pre-determined conditions, several of which are described herein below. Various types of the deflector element, featuring different geometrical shapes, configurations, sizes, and, exhibiting desirable properties, may be constructed for fulfilling the following described conditions.

When testing the deflecting device of the present invention under physiological conditions in the carotid region of a subject, namely:

Reₐᵥ=200-500,

BPM (heart beats per minute)=40-180,

Womersley=2-7,

wherein Reₐᵥ is the average Reynolds number of the blood flowing at an arterial bifurcation of the carotid region, and, Womersley is the dimensionless heart beat parameter, the following conditions should preferably be met by the deflector element, of the coil element of the deflecting device of the present invention:

(1) Reₚᵣₒₓ is, preferably, in a range of between about 0.3 to about 30, and, more preferably, in a range of between 0 and about 4, and, is also, preferably, equal to or less than 1, in accordance with creeping or Stokes' flow, and,

(2) Shear Stress is in a range of between less than about 100 dyne/cm² and greater than about 2 dyne/cm²,

wherein Reₚᵣₒₓ is the Reynolds number for a single wire of which the deflector element is made, and, the shear stress is measured at the deflecting device. As known to a person having ordinary skill in the art, the smaller Reₚᵣₒₓ is, the better the performance of the deflecting device. However, the deflecting device may also operate at larger values of Reₚᵣₒₓ than indicated above, whereby, the present invention is by no means limited to any specific value of Reₚᵣₒₓ.

The deflecting device according to the present invention is useful in a variety of cases. Some illustrative indications are listed below:

(1) Embolic strokes from proximal sources. These are:

Atrial fibrillation (2.5 million in the U.S.A. in 1999);

Mechanical heart valve (225,000 procedures performed annually in the U.S.);

Subjects at high risk for recurrent embolism for a certain period (S.B.E.);

Subjects at high risk for proximal emboli and absolute contraindications for anticoagulation;

Subjects at high risk for proximal emboli failing best medical treatment.

(2) In cases in where carotid stents are introduced to treat local stenosis, it is possible to introduce and deploy the deflecting device of the present invention during the same procedure if there are concomitant high risk proximal sources of emboli. These are, for instance:

Protruding Aortic arch atheroma (more than ⅓ of symptomatic subjects);

Severe carotid stenosis with concomitant cardiac disease;

Severe carotid stenosis in subjects undergoing heart surgery (5% on the statistical basis of 600,000 coronary bypass surgeries).

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

While the invention has been described in conjunction with specific embodiments and examples thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. An implantable device implantable in an artery of a patient at a bifurcation thereof into a first branch supplying blood to a vital region having a high sensitivity to emboli in the blood, and a second branch supplying blood to a less vital region having a lower sensitivity to emboli in the blood;
said implantable device being of tubular configuration initially of a small diameter for facilitating its introduction into and deployment through the artery to said bifurcation, and expandable to a larger diameter for implantation in the artery and said bifurcation;
said implantable device comprising: a base element configured and dimensioned for anchoring said implantable device in the artery at said bifurcation; and a deflector element configured and dimensioned for covering the inlet of said first branch at the bifurcation when said implantable device is implanted in said artery;
said deflector element being formed with openings therethrough of a size and configuration to deflect emboli in the blood to said second branch without blocking blood flow through said second branch or through said first branch;
said base element including an anchoring portion engageable with the inner surface of the artery for anchoring the implantable device therein, and a supporting portion engageable with a surface of said deflector element for fixing said deflector element over said inlet of said first branch at said bifurcation when the implantable device is anchored in said artery;
said deflector element being attached to said supporting portion of the base element to produce a composite construction.

2. The implantable device according to claim 1, wherein said base element is a coil having two opposing ends which overlap in its initial small diameter condition.

3. The implantable device according to claim 2, wherein said coil is a perforated sheet coiled into said tubular configuration.

4. The implantable device according to claim 1, wherein said base element is a tube in its initial small-diameter condition expandable to said larger-diameter condition.

5. The implantable device according to claim 1, wherein said supporting portion of the base element includes a plurality of spaced parallel filaments extending longitudinally of said base element.

6. The implantable device according to claim 5, wherein said supporting portion of the base element further includes a pair of additional filaments extending circumferentially of said base element on opposite ends of said supporting portion.

7. The implantable device according to claim 5, wherein said deflector element is supported on, and is draped between, said plurality of spaced parallel filaments of the supporting portion of the base element in the small-diameter condition of the implantable device.

8. The implantable device according to claim 5, wherein said deflector element is supported on, and is stitched to, said plurality of spaced parallel filaments of the supporting portion of the base element in the small-diameter condition of the implantable device.

9. The implantable device according to claim 5, wherein said deflector element is supported on, and is wrapped around, said plurality of spaced parallel filaments of the supporting portion of the base element in the small-diameter condition of the implantable device.

10. The implantable device according to claim 1, wherein said deflector element includes a finely-meshed area circumscribed by an unmeshed frame.

11. The implantable device according to claim 1, wherein said base element and said deflector element are both of a meshed structure in which the meshed structure of the base element has a larger porosity index than that of the deflector element.

12. The implantable device according to claim 1, wherein said implantable device is configured and dimensioned for implantation in the patient's common carotid artery at its bifurcation with the internal carotid artery constituting said first branch, and the external carotid artery constituting said second branch.

13. The implantable device according to claim 1, wherein said openings in the deflector element are within the range of 100 μm to 700 μm.

14. The implantable device according to claim 1, wherein said openings in the deflector element are within the range of 100 μm to 400 μm.

15. The implantable device according to claim 1, wherein said base element is constituted of wire of a diameter within the range of 100 μm to 1500 μm.

16. The implantable device according to claim 1, wherein said base element is constituted of wire of a diameter within the range of 100 μm to 200 μm.

17. The implantable device according to claim 15 wherein said deflector element is constituted of wire of a diameter within the range of 20 μm to 75 μm.

18. The implantable device according to claim 15 wherein said deflector element is constituted of wire of a diameter within the range of 20 μm to 40 μm.

19. A method of reducing the risk of stroke in a patient, comprising:
providing an implantable device according to claim 1 configured and dimensioned for implantation in the patient's common carotid artery at its bifurcation with the internal carotid artery constituting said first branch, and the external carotid artery constituting said second branch;
and implanting said implantable device in the patient's common carotid artery at said bifurcation.

20. A method for assembling an implantable device implantable in an artery of an individual at a bifurcation thereof into a first branch supplying blood to a vital region having a high sensitivity to emboli in the blood, and a second branch supplying blood to a less vital region having a lower sensitivity to emboli in the blood:
selecting a base element of tubular configuration, said base element having states of different diameter, a first state being a small diameter for facilitating its introduction into and deployment through the artery to the bifurcation, and expandable to a second state being a larger diameter for implantation in the artery and said bifurcation;
selecting a separate tubular deflector element, comprised of elastic elements, said tubular deflector element being configured and dimensioned to cover the inlet of the first branch at said bifurcation when the implantable device is implanted in said artery, the unstretched diameter of said tubular deflector element being smaller than said diameter of said second state of said base element;
inserting said base element in a contracted condition into said tubular deflector element;
contracting said base element and said tubular deflector element to said first state thus facilitating its introduction into and deployment through the patient body to said bifurcation; and
expanding said base element to said second state thus implantating said implantable device in the artery at said bifurcation and securing said separate tubular deflector element to said base element.
